(19) United States
(12) Patent Application Publication
(10) Pub. No.: US 2004/0019101 A1
(43) Pub. Date: Jan. 29, 2004

(54) Methods of treating gastrointestinary and genitourinary pain disorders

(75) Inventors: Robyn Gail Karlstadt, Cherry Hill, NJ (US); Richard Brian Lynn, Bala Cynwyd, PA (US); Michael Scott Burton, Lansdale, PA (US); Mervyn Danilewitz, Villanova, PA (US)

Correspondence Address: WYETH, PATENT LAW GROUP, FIVE GIRALDA FARMS, MADISON, NJ, 07940, US

(73) Assignee: Wyeth, Madison, NJ

(21) Appl. No.: 10/438,572

(22) Filed: May 15, 2003

Related U.S. Application Data
(60) Provisional application No. 60/381,305, filed on May 17, 2002.

(51) Int. Cl. A61K 31/36; A61K 31/277; A61K 31/137

(52) U.S. Cl. 514/464; 514/524; 514/649

(57) ABSTRACT

This invention provides a method of treating functional gastrointestinal and genitourinary disorders in a mammal by administering to the mammal an effective amount of a hydroxycycloalkane phenethylamine of the following structural formula:

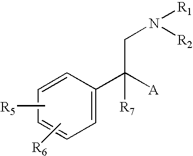

in which A is a moiety of the formula

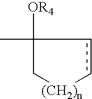

where

the dotted line represents optional unsaturation;

R₁ is hydrogen or alkyl;

R₂ is alkyl;

R₄ is hydrogen, alkyl, formyl, or alkanol;

R₅ and R₆ are, independently, hydrogen, hydroxyl, alkyl, alkoxy, alkanoyloxy, cyano, nitro, alkylmercapto, amino, alkylamino, dialkylamino, alkanamido, halo, trifluoromethyl, or taken together, methylene dioxy;

R₇ is hydrogen or alkyl; and

n is 0, 1, 2, 3, or 4;

or a pharmaceutically acceptable salt thereof.

This application claims priority from co-pending provisional application serial No. 60/381,305, filed on May 17, 2002, the entire disclosure of which is hereby incorporated by reference.

### BACKGROUND OF THE INVENTION

(1-[2-(dimethylamino)-1-(4-methoxyphenyl) ethyl] cyclohexanol), or therapeutically acceptable salts thereof, known generally as venlafaxine, and its analogues are disclosed in U.S. Pat. No. 4,535,186 (Husbands et al.). These compounds have been previously reported to be useful as an antidepressant. U.S. Pat. No. 4,535,186 teaches the production of venlafaxine and its analogues and is incorporated herein as reference.

Venlafaxine and its active metabolite, O-desmethyl venlafaxine, have been shown to be potent inhibitors of monoamine neurotransmitter uptake, a mechanism associated with clinical antidepressant activity. Due to its novel structure, venlafaxine has a mechanism of action unrelated to other available antidepressants, such as the tricyclic antidepressants desipramine, nostriptyline, protriptyline, imipramine, amitryptyline, trimipramine and doxepin.

It is believed that venlafaxine's mechanism of action is related to potent inhibition of the uptake of the monoamine neurotransmitters serotonin and norepinephrine. To a lesser degree, venlafaxine also inhibits dopamine reuptake, but it has no inhibitory activity on monoamine oxidase. O-desmethylvenlafaxine, venlafaxine's major metabolite in humans, exhibits a similar pharmacologic profile. Venlafaxine's ability to inhibit norepinephrine and serotonin (5-HT) uptake has been predicted to have an efficacy which rivals or surpasses that of tricyclic antidepressants (Stuart A. Montgomery, M. D., J. Clin. Psychiatry, 54:3, March 1993).

In contrast to classical tricyclic antidepressant drugs, venlafaxine has virtually no affinity for muscarinic, histaminergic or adrenergic receptors in vitro. Pharmacologic activity at these receptors is associated with the various anticholinergic, sedative and cardiovascular effects seen with the tricyclic antidepressant drugs.

Functional gastrointestinal and gastrourinary disorders include irritable bowel syndrome, symptomatic GERD, hypersensitive esophagus, nonulcer dyspepsia, noncardiac chest pain, biliary dyskinesia, sphincter of oddi dysfunction, interstitial cystitis (irritable bladder), and chronic pelvic pain (including, but not limited to vulvodynia, prostatodynia and proctalgia).

Functional gastrointestinal and genitourinary disorders are chronic disorders for which no specific structural, biochemical or infectious etiology has been found.

Irritable bowel syndrome, also known as “spastic colon” is a common disorder of the colon and small intestine defined by symptoms of abdominal pain and altered bowel habits. Patients with IBS typically complain of diarrhea alternating with constipation although some patient experience predominance of one or the other. Other symptoms that are more common in IBS than in other gastrointestinal disorders include abdominal distention, pain relief with bowel movement, more frequent stools with the onset of pain, looser stools with the onset of pain, passage of mucus, and the sensation of incomplete evacuation.

Nonulcer dyspepsia is a functional disorder of the gastroduodenum and is characterized by persistent or recurrent feelings of upper abdomen discomfort or pain which is not associated with diarrhea or constipation. Discomfort is a negative feeling characterized by one or more of several symptoms including early satiety, postprandial fullness or bloating.

Noncardiac chest pain patients frequently experience replication of their pain with smaller volumes of esophageal balloon distention than those required to produce pain in asymptomatic persons. Visceral hypersensitivity may contribute to the patients interpretation of pain.

Patients with biliary dyskinesia having right upper quadrant pain or epigastric pain which may be disabling and lasts for minutes to hours. The pain may be continuous with intermittent exacerbations. The pain may radiate to the back or shoulders and may be accompanied by nausea and vomiting.

IBS patients account for 12% of visits to primary care physicians and 25-50% of visits to gastroenterologists. Although IBS is believed benign, it is a chronic recurrent disorder that significantly impacts quality of life and is associated with high direct costs including medical visits, investigations, medications and lost work time.

Tricyclic antidepressants such as amytriptiline, doxepin and imipramine have been demonstrated to be efficacious for the treatment of irritable bowel syndrome. However, the use of TCAs is limited by side effects such as sedation and constipation and concerns about safety.

Treatment of IBS with an SSRI have also been reported. However, SSRI's do not appear to affect whole gut transition times either in healthy subjects or IBS patients compared to TCAs such as imipramine which prolong orocecal transit.

### DESCRIPTION OF INVENTION

In accordance with the present invention there is provided a method of treating, preventing, or controlling function gastrointestinal disorders including irritable bowel syndrome, chronic abdominal pain and nonulcer dyspepsia and accompanying symptoms in mammals, preferably in humans.

The methods of the present invention involve administering to a mammal in need thereof an effective amount of one or more compounds from a group of substituted phenethylamines. The compounds of this invention present the following structural formula:

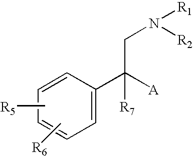

in which A is a moiety of the formula

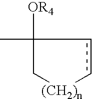

where

the dotted line represents optional unsaturation;

R₁ is hydrogen or alkyl of 1 to 6 carbon atoms;

R₂ is alkyl of 1 to 6 carbon atoms;

R₄ is hydrogen, alkyl of 1 to 6 carbon atoms, formyl, or alkanol of 2 to 7 carbon atoms;

R₅ and R₆ are independently hydrogen, hydroxyl, alkyl of 1 to 6 carbon atoms, alkoxy of 1 to 6 carbon atoms, alkanoyloxy of 2 to 7 carbon atoms, cyano, nitro, alkylmercapto of 1 to 6 carbon atoms, amino, alkyl-amino of 1 to 6 carbon atoms, dialkylamino in which each alkyl group is of 1 to 6 carbon atoms, alkanamido of 2 to 7 carbon atoms, halo, trifluoromethyl, or when taken together, methylene dioxy;

R₇ is hydrogen or alkyl of 1 to 6 carbon atoms; and n is one of the integers 0, 1, 2, 3, or 4;

or a pharmaceutically acceptable salt thereof.

More preferred compounds useful in methods of the present invention are those of the formula:

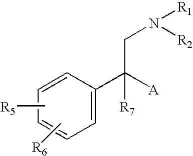

in which

A is as defined supra;

R₁ is hydrogen or alkyl of 1 to 3 carbon atoms;

R₂ is alkyl of 1 to 3 carbon atoms;

R₅ is hydrogen, hydroxyl, alkoxy of 1 to 3 carbon atoms, chloro, bromo, trifluoromethyl or alkyl of 1 to 3 carbon atoms;

R₆ is alkyl of 1 to 3 carbon atoms, alkoxy of 1 to 3 carbon atoms, chloro, bromo, trifluoromethyl or alkanoyloxy of 2 to 3 carbon atoms;

R₇ is hydrogen or alkyl of 1 to 3 carbon atoms;

or a pharmaceutically acceptable salt thereof.

The most preferred compounds useful in methods of the present invention are those in which R₅ and R₆ are both in the meta positions or one of R₅ or R₆ is in the para position and n is 2.

Of particular interest are the compounds 1-[(2-dimethylamino)-1-(4-methoxyphenyl)ethyl]cyclohexanol and 1-[(2-dimethylamino)-1-(4-hydoxyphenyl)ethyl]cyclohexanol and the enantiomers and pharmaceutically acceptable salts thereof.

The compounds in which R₄ is formyl or alkanoyl of 2 to 7 carbon atoms have been found to be not as potent as the corresponding free hydroxy bearing derivatives. However, in long term therapy the acyloxy derivatives will act as pro drugs as the acyl group is removed in vivo either via acid hydrolysis in the stomach or enzymatically.

The pharmaceutically acceptable acid addition salts of the basic compounds of this invention are formed conventionally by reaction of the free base with an equivalent amount of any acid which forms a non-toxic salt. Illustrative acids are either inorganic or organic, including hydrochloric, hydrobromic, fumaric, maleic, succinic, sulfuric, phosphoric, tartaric, acetic, citric, oxalic and similar acids. For parenteral administration, the use of water soluble salts is preferred, although either the free base of the pharmaceutically acceptable salts are applicable for oral or parenteral administration of the antidepressant agents of this invention. The halo substituent representing R₅ or R₆ is intended to include the chloro, bromo, iodo or fluoro substituents.

Pharmaceutical compositions containing the compounds of this invention may be administered to subjects in accordance with the invention. The active ingredient can be compounded into any of the usual oral dosage forms including tablets, capsules and liquid preparations such as elixirs and suspensions containing various coloring, flavoring, stabilizing and flavor masking substances. For compounding oral dosage forms, the active ingredient can be mixed with various conventional tableting materials such as starch, calcium carbonate, lactose, sucrose and dicalcium phosphate to aid the tableting or capsulating process. Magnesium stearate, as an additive, provides a useful lubricant function when desired.

The active ingredients can be dissolved or suspended in a pharmaceutically acceptable sterile liquid carrier, such as sterile water, sterile organic solvent or a mixture of both. Preferably a liquid carrier is one suitable for parenteral injection. Where the active ingredient is sufficiently soluble it can be dissolved in normal saline as a carrier; if it is too insoluble for this it can often be dissolved in a suitable organic solvent, for instance aqueous propylene glycol or polyethylene glycol solutions. Aqueous propylene glycol containing from 10 to 75% of the glycol by weight is generally suitable. In other instances other compositions can be made by dispersing the finely-divided active ingredient in aqueous starch or sodium carboxymethyl cellulose solution, or in a suitable oil, for instance arachis oil. Liquid pharmaceutical compositions which are sterile solutions or suspensions can be utilized by intramuscular, intraperitoneal or subcutaneous injection.

Preferably the pharmaceutical composition is in unit dosage form, e.g. as tablets or capsules. In such form, the composition is sub-divided in unit doses containing appropriate quantities of the active ingredient; the unit dosage forms can be packaged compositions, for example, packeted powders or vials or ampoules. The unit dosage form can be a capsule, cachet or tablet itself, or it can be the appropriate number of any of these in package form. The quantity of the active ingredient in a unit dose of composition may be varied or adjusted from 2 mg. or less to 50 mg. or more, according to the particular need and the activity of the active ingredient. The usual oral recommended dose of venlafaxine for humans may be between about 75 and about 200 mg/day and this dose may be administered in two or three divided doses, preferably with food if administered orally. A maximum recommended daily dose for humans would be about 375 mg, but it will be understood by one skilled in the art that dosage under this invention will be determined by the particular circumstances surrounding each case.

One skilled in this art will also be aware that the routes of administering the compounds of this invention may vary significantly. In addition to other oral administrations, sustained release compositions may be favored. Other acceptable routes may include, but are not limited to, intravenous, intramuscular and intraperitoneal injections, subdermal implants, as well as buccal, sublingual, transdermal, topical, rectal, vaginal and intranasal administrations. Bioerodible, non-bioerodible, biodegradable and non-biodegradable systems of administration may also be used.

It should also be understood that the present invention is intended to include all methods of, and reasons for, treating symptoms of irritable bowel syndrome in mammals, preferably in humans. For the purposes of this invention, treating irritable bowel syndrome is to be understood as including all prophylactic, therapeutic, progression inhibiting, remedial, maintenance, curative or other treatments, regimens or administrations of or with venlafaxine that yield the desired effects in the mammal receiving compounds of the invention.

What is claimed is:

1. A method of treating a functional gastrointestinal or genitourinary disorder in a mammal, comprising administering to the mammal an effective amount of a compound of the formula: 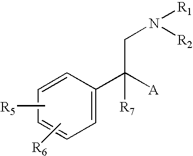
in which A is a moiety of the formula 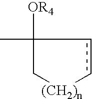
wherein
the dotted line represents optional unsaturation;
R₁ is hydrogen or alkyl of 1 to 6 carbon atoms;
R₂ is alkyl of 1 to 6 carbon atoms;
R₄ is hydrogen, alkyl of 1 to 6 carbon atoms, formyl, or alkanol of 2 to 7 carbon atoms;
R₅ and R₆ are, independently, hydrogen, hydroxyl, alkyl of 1 to 6 carbon atoms, alkoxy of 1 to 6 carbon atoms, alkanoyloxy of 2 to 7 carbon atoms, cyano, nitro, alkylmercapto of 1 to 6 carbon atoms, amino, alkylamino of 1 to 6 carbon atoms, dialkylamino in which each alkyl group is of 1 to 6 carbon atoms, alkanamido of 2 to 7 carbon atoms, halo, trifluoromethyl, or taken together, methylene dioxy;
R₇ is hydrogen or alkyl of 1 to 6 carbon atoms; and
n is 0, 1, 2, 3, or 4;
or a pharmaceutically acceptable salt thereof.

2. The method of claim 1 wherein the compound is: 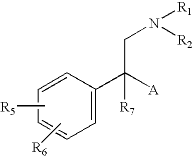
in which A is a moiety of the formula 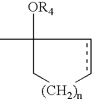
wherein
the dotted line represents optional unsaturation, and
R₁ is hydrogen or alkyl of 1 to 3 carbon atoms;
R₂ is alkyl of 1 to 3 carbon atoms;
R₅ is hydrogen, hydroxyl, alkoxy of 1 to 3 carbon atoms, chloro, bromo, trifuoromethyl or alkyl of 1 to 3 carbon atoms;
R₆ is alkyl of 1 to 3 carbon atoms, alkoxy of 1 to 3 carbon atoms, chloro, bromo, trifluoromethyl or alkanoyloxy of 2 to 3 carbon atoms.
R₇ is hydrogen or alkyl of 1 to 3 carbon atoms;
or a pharmaceutically acceptable salt thereof.

3. The method of claim 2 wherein R₅ and R₆ are both in the meta positions or one of R₅ or R₆ is in the para position and n is 2.

4. The method of claim 2 wherein the compound is 1-[(2-dimethylamino)-1-(4-methoxyphenyl)ethyl]cyclohexanol or a pharmaceutically acceptable salt thereof.

5. The method of claim 2 wherein the compound is 1-[2-(dimethylamino)-1-(4-hydroxyphenyl)ethyl]cyclohexanol or a pharmaceutically acceptable salt thereof.

6. The method of claim 1 wherein the effective amount comprises a daily dose of between 35 mg/day to about 75 mg/day.

7. The method of claim 1 wherein the effective amount comprises a daily dose of between about 50 mg/day and about 375 mg/day.

8. The method of claim 1 wherein the effective amount comprises a daily dose of between about 75 mg/day and about 200 mg/day.

9. The method of claim 1 wherein the subject is a human.

10. The method of claim 1 wherein the functional gastrointestinal disorder is irritable bowel syndrome.

11. The method of claim 1 wherein the functional gastrointestinal disorder is symptomatic GERD.

12. The method of claim 1 wherein the functional gastrointestinal disorder is hypersensitive esophagus.

13. The method of claim 1 wherein the functional gastrointestinal disorder is nonulcer dyspepsia.

14. The method of claim 1 wherein the functional gastrointestinal disorder is noncardiac chest pain.

15. The method of claim 1 wherein the functional gastrointestinal disorder is biliary dyskinesia.

16. The method of claim 1 wherein the functional gastrointestinal disorder is sphincter of oddi dysfunction.

17. The method of claim 1 wherein the functional genitourinary disorder is chronic pelvic pain.

18. The method of claim 1 wherein the functional genitouriniary disorder is interstitial cystitis.
